(19) United States
(12) Patent Application Publication
(10) Pub. No.: US 2019/0350443 A1
(43) Pub. Date: Nov. 21, 2019

(54) Method For Monitoring A Property Of Tissue Of An Internal Bodily Organ And Adjusting The Tissue Property

(71) Applicant: Mark D. Noar, Owings Mills, MD (US)

(72) Inventor: Mark D. Noar, Owings Mills, MD (US)

(21) Appl. No.: 16/417,948

(22) Filed: May 21, 2019

Related U.S. Application Data
(60) Provisional application No. 62/674,121, filed on May 21, 2018.

(51) Int. Cl. A61B 1/012; A61B 5/145; A61B 5/00; A61B 1/00

(52) U.S. Cl. CPC A61B 1/012; A61B 5/14539; A61B 1/00016; A61B 5/4839; A61B 5/6847

(57) ABSTRACT

A method monitors a property of tissue of an internal bodily organ that is not functioning properly or that is threated by disease. The method monitors, in real time, the property of tissue of the internal bodily organ by contacting the tissue. Based on the monitoring step, the method adjusts the property of the tissue to improve functioning of the internal bodily organ, to help prevent further damage to the tissue, to prevent death of the tissue, or to help the tissue recover from a diseased state. A device for monitoring and adjusting a property of tissue of an internal bodily organ is also provided.

### BACKGROUND

U.S. Pat. No. 7,736,320 hereby incorporated by reference in its entirety herein, discloses a system for monitoring a patient's breath chemistry using a self-condensing pH sensor inserted into the nasal channels. This device and method is used in the oropharynx region where there is dry or moist gaseous conditions to diagnose certain respiratory diseases associated with gastroesophageal reflux (GER). Thus, the pH sensor is used to monitor pH at a location that is remote from the location causing the GER.

Although the conventional method and system is well suited for a merely diagnostic purpose, there is a need to monitor a property of tissue of internal bodily organs that are not functioning properly or threatened by disease and to adjust the property of the tissue to promote improved functioning or healing of the organ.

### SUMMARY

An objective of the embodiment is to fulfill the need referred to above. In accordance with an aspect of an embodiment, a method monitors a property of tissue of an internal bodily organ that is not functioning properly or that is threated by disease. The method monitors, in real time, the property of tissue of the internal bodily organ by contacting the tissue. Based on the monitoring step, the method adjusts the property of the tissue to improve functioning of the internal bodily organ, to help prevent further damage to the tissue, to prevent death of the tissue, or to help the tissue recover from a diseased state.

In accordance with another aspect of an embodiment, a device for monitoring and adjusting a property of tissue of an internal bodily organ includes a probe constructed and arranged to contact the tissue to sense the property of the tissue and a delivery structure constructed and arranged to deliver a substance to the tissue to adjust the property of the tissue.

Other objectives, features and characteristics of the present embodiment, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawing, wherein like reference numerals refer to like parts, in which:

FIG. 1 a partial sectional view of a human head and showing a first probe for monitoring brain tissue and a second probe for monitoring bronchial tissue in accordance with embodiments of the invention.

FIG. 2 is a flow chart showing steps of a method of an embodiment.

### DETAILED DESCRIPTION OF EXAMPLE EMBODIMENTS

The present invention relates to a device and method for monitoring a property of tissue of an internal bodily organ that is not functioning properly or that is threatened by disease and to adjust the property of the tissue to promote proper functioning or healing and recovery of the organ. The inventor determined that conventional pH probes can be used inside the brain in patients with brain injury. The inventor's research shows that by monitoring and adjusting pH in brain tissue, one can save the life of a patient what has been up until now a uniformly fatal condition, salvaging approximately 50% of patients by being able to monitor and adjust pH inside the brain.

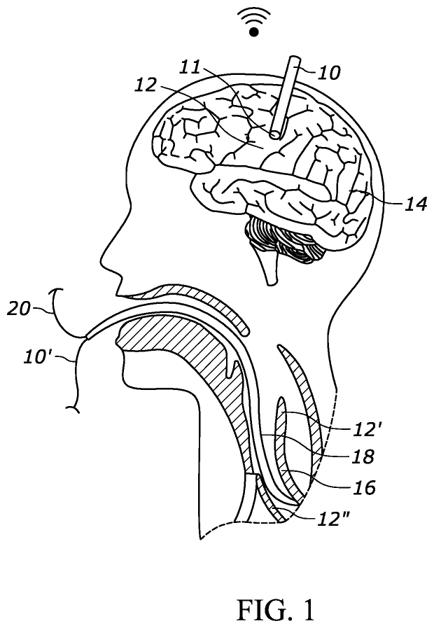

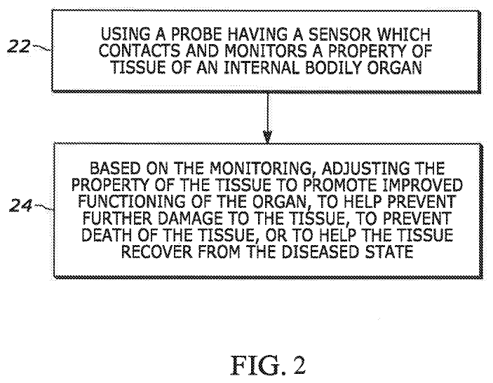

Thus, with reference to FIGS. 1 and 2, a method of monitoring, in real time, a property of tissue of an internal bodily organ and, based on the monitoring, adjusting the property thereof will be appreciated. The method, in step 22 of FIG. 2, preferably includes using a probe 10 (FIG. 1) having a sensor 11 that contacts and thus monitors the property of tissue 14 of an internal bodily organ, such as the brain 12. In step 24, based on the monitoring, if needed, the property of the tissue that was monitored is adjusted to promote improved functioning of the organ, to help prevent further damage to the tissue, to prevent death of the tissue, or to help the tissue recover from the diseased state. In the embodiment, the property of the tissue is pH level, the sensor 11 is a pH sensor, and the step of adjusting the tissue property includes adjusting the pH level of the tissue, for example, so as to return the pH level to a level of a normally functioning organ.

Also shown in FIG. 1 is another embodiment of the method wherein the probe 10′, to monitor the property of tissue 14 of an internal bodily organ such as the tracheal 12′, or bronchial tube 12″ is employed through a catheter or bronchoscope or endoscope 18 The probe 10′ is shown in position to contact the bronchial tissue to monitor, measure and maintain pH of the bronchial tissue and therefore help control and reverse severe pulmonary disease.

The method disclosed herein can be applied to any internal bodily tissue that has an essential property indicative of disease or malfunction of an organ, which can be contacted, monitored and adjusted. Preferably, the bodily tissue is from an organ that does not contain gaseous pH. For example, in addition to the two examples of FIG. 1, the tissue can also be spinal cord tissue, thecal tissue, bladder tissue, lung tissue, kidney tissue, testicle tissue, ovary tissue or other bodily tissues.

The method can employ any probe 10, 10′ that can contact and sense the property being monitored. Properties that can be monitored are, for example, pH level, electrolyte level, protein level, hormone level, estrogen or testosterone levels, or any other essential chemical level that indicates that the tissue is damaged or is not functioning properly. For example, if pH is being monitored, the probe can be of type disclosed in U.S. Pat. No. 7,736,320. Although the probe preferably does not damage tissue and monitors the tissue at the tissue site, the probe can be configured to obtain a biopsy of the tissue so that the tissue can be analyzed remotely from the tissue site for the property of interest and thereafter, if needed, adjustments of the tissue property can be made.

The probe 10 of FIG. 1 is preferably wireless and can obtain information from the tissue 12 and then communicate the data in a wireless manner to a portable device (not shown) for analyzing the data. The probe 10 can instead be a wired device that is directly electrically connected to a data receiver or recorder (not shown). The probe 10, 10′ is shown to be used and then removed from the patient. Alternatively, the probe can be configured to be implanted so as to monitor the tissue continuously over a time period and to transmit the data wirelessly to a device external to the body. Although the term “probe” is used herein, the probe can be of any shape or configuration suitable for obtaining information regarding the property of the tissue to be monitored.

As shown in FIG. 1, a delivery structure such as a lumen can be provided through the endoscope 18 containing the probe 10′ so as to deliver a substance, for example, a compound or chemical that increases or decreases acidity or alkalinity so as to adjust the pH level of the tissue. The substance can thus be delivered directly to the tissue without having to remove the probe 10′. In this way, the pH level can be monitored simultaneously as the adjustment of the tissue property is made. Alternatively, the monitored property (e.g., pH) can be adjusted without using the probe 10, 10′. For example, a substance can be injecting into the bloodstream (or otherwise introduced to the bloodstream) that reaches the tissue of the organ being monitored so as to adjust the property of the tissue.

Thus, unlike conventional diagnostic procedures that sense a bodily condition or property at a location that is remote from source causing a bodily issue, the method of the embodiment can sense the bodily condition or property directly at the tissue of the diseased organ and based on the sensing, one can adjust the property of the tissue to improve the functioning of the bodily tissue or organ, to help prevent further damage to the tissue, to prevent death of the tissue, or to help the tissue recover from the diseased state.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. A method of monitoring a property of tissue of an internal bodily organ that is not functioning properly or that is threated by disease, the method comprising:
monitoring, in real time, the property of tissue of the internal bodily organ by contacting the tissue, and
based on the monitoring step, adjusting the property of the tissue to improve functioning of the internal bodily organ, to help prevent further damage to the tissue, to prevent death of the tissue, or to help the tissue recover from a diseased state.

2. The method of claim 1, wherein the step of adjusting the property of the tissue includes returning the property to a normal state or preventing further deterioration of the tissue.

3. The method of claim 2, wherein the tissue is one of brain tissue, spinal cord tissue, thecal tissue, bronchial tissue, tracheal tissue, lung tissue, kidney tissue, testicle tissue, bladder tissue and ovary tissue, or other tissues of the body.

4. The method of claim 1, wherein the property of the tissue is pH level, or other essential tissue properties and the step of delivering therapy includes adjusting a pH level or other essential level of the tissue.

5. The method of claim 1, wherein the step of monitoring the property of the tissue includes:
providing a probe having a sensor to sense the property of the tissue, and
contacting the tissue with the sensor to obtain data regarding the property of the tissue.

6. The method of claim 5, wherein the step of adjusting the property of the tissue occurs while the probe remains at the tissue site.

7. The method of claim 1, wherein the step of adjusting the property of the issue is performed during the monitoring step.

8. The method of claim 5, wherein the step of adjusting the property of the tissue occurs after the sensor is removed from contact with the tissue.

9. The method of claim 1, wherein the step of adjusting the property of the tissue includes introducing a substance into a patient's bloodstream so that when the substance reaches the tissue, the substance adjusts the property of the tissue.

10. The method of claim 1, wherein the step of adjusting the property of the tissue includes adjusting the property directly at the monitored tissue.

11. The method of claim 5, wherein the probe is constructed and arranged to transmit the data in a wireless manner.

12. A device for monitoring and adjusting a property of tissue of an internal bodily organ, the device comprising:
a probe constructed and arranged to contact the tissue to sense the property of the tissue; and
a delivery structure constructed and arranged to deliver a substance to the tissue to adjust the property of the tissue.

13. The device of claim 12, wherein the delivery structure is a lumen.

14. The device of claim 12, in combination with an endoscope, the probe and delivery structure being disposed in the endoscope.

15. The device of claim 12, wherein the probe is constructed and arranged to deliver data regarding the sensed property in a wireless manner.

16. The device of claim 15, wherein probe includes a sensor to sense the pH level as the property of the tissue, and the delivery structure is constructed and arranged to deliver the substance to adjust the pH level of the tissue.
